SEQUENCE LISTING (12 sequences)

SEQ ID NO: 1 - DNA, 636 bp, Mus musculus
atgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctag

SEQ ID NO: 2 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 3 - DNA, 636 bp, Rattus norvegicus
atgaagtttctctccgcaagagacttccagccagttgccttcttgggactgatgttgttgacagccactgccttccctacttcacaagtccggagaggagacttcacagaggataccacccacaacagaccagtatataccacttcacaagtcggaggcttaattacatatgttctcagggagatcttggaaatgagaaaagagttgtgcaatggcaattctgattgtatgaacagcgatgatgcactgtcagaaaacaatctgaaacttccagaaatacaaagaaatgatggatgcttccaaactggatataaccaggaaatttgcctattgaaaatctgctctggtcttctggagttccgtttctacctggagtttgtgaagaacaacttacaagataacaagaaagacaaagccagagtcattcagagcaatactgaaaccctagttcatatcttcaaacaagagataaaagactcatataaaatagtccttcctaccccaacttccaatgctctcctaatggagaagttagagtcacagaaggagtggctaaggaccaagaccatccaactcatcttgaaagcacttgaagaatttctaaaggtcactatgaggtctactcggcaaacctag

SEQ ID NO: 4 - protein, 211 aa, Rattus norvegicus
MKFLSARDFQPVAFLGLMLLTATAFPTSQVRRGDFTEDTTHNRPVYTTSQVGGLITYVLREILEMRKELCNGNSDCMNSDDALSENNLKLPEIQRNDGCFQTGYNQEICLLKICSGLLEFRFYLEFVKNNLQDNKKDKARVIQSNTETLVHIFKQEIKDSYKIVLPTPTSNALLMEKLESQKEWLRTKTIQLILKALEEFLKVTMRSTRQT

SEQ ID NO: 5 - DNA, 639 bp, Human
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 6 - protein, 212 aa, Human
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 7 - DNA, 1323 bp, Mus musculus
atgctgaccgtcggctgcacgctgttggtcgccctgctggccgcgcccgcggtcgcgctggtcctcgggagctgccgcgcgctggaggtggcaaatggcacagtgacaagcctgccaggggccaccgttaccctgatttgccccgggaaggaagcagcaggcaatgttaccattcactgggtgtactctggctcacaaaacagagaatggactaccacaggaaacacactggttctgagggacgtgcagctcagcgacactggggactatttatgctccctgaatgatcacctggtggggactgtgcccttgctggtggatgttcccccagaggagcccaagctctcctgcttccggaagaacccccttgtcaacgccatctgtgagtggcgtccgagcagcaccccctctccaaccacgaaggctgtgctgtttgcaaagaaaatcaacaccaccaacgggaagagtgacttccaggtgccctgccagtattctcagcagctgaaaagcttctcctgccaggtggagatcctggagggtgacaaagtataccacatagtgtcactgtgcgttgcaaacagtgtgggaagcaagtccagccacaacgaagcgtttcacagcttaaaaatggtgcagccggatccacctgccaaccttgtggtatcagccatacctggaaggccgcgctggctcaaagtcagctggcagcaccctgagacctgggacccgagttactacttgctgcagttccagcttcgataccgacctgtatggtcaaaggagttcacggtgttgctgctcccggtggcccagtaccaatgcgtcatccatgatgccttgcgaggagtgaagcacgtggtccaggtccgtgggaaggaggagcttgaccttggccagtggagtgaatggtccccagaggtcacgggcactccttggatagcagagcccaggaccaccccggcaggaatcctctggaaccccacacaggtctctgttgaagactctgccaaccacgaggatcagtacgaaagttctacagaagcaacgagtgtcctcgccccagtgcaagaatcctcgtccatgtccctgcccacattcctggtagctggaggaagcttggcgtttgggttgcttctctgtgtcttcatcatcctgtgttgggagccgcgcccacattcgccgttacaagatggcgctgacagctgtgttctaagtggtaaacaaataatctgcgcatgtgccgagggtggttctccactccatgtgctctgccttccccgtgacgtcaactcggccgatgggctgcagccaatcagggagtga

SEQ ID NO: 8 - protein, 364 aa, Mus musculus
MLTVGCTLLVALLAAPAVALVLGSCRALEVANGTVTSLPGATVTLICPGKEAAGNVTIHWVYSGSQNREWTTTGNTLVLRDVQLSDTGDYLCSLNDHLVGTVPLLVDVPPEEPKLSCFRKNPLVNAICEWRPSSTPSPTTKAVLFAKKINTTNGKSDFQVPCQYSQQLKSFSCQVEILEGDKVYHIVSLCVANSVGSKSSHNEAFHSLKMVQPDPPANLVVSAIPGRPRWLKVSWQHPETWDPSYYLLQFQLRYRPVWSKEFTVLLLPVAQYQCVIHDALRGVKHVVQVRGKEELDLGQWSEWSPEVTGTPWIAEPRTTPAGILWNPTQVSVEDSANHEDQYESSTEATSVLAPVQESSSMSLP

SEQ ID NO: 9 - DNA, 1389 bp, Rattus norvegicus
atgctggccgtcggctgcaccctgctggtcgccctgctggccgcgcccgcagtcgcgctggtccttgggagctgccgcgcgctggaggtggcaaatggtacggtgacgagcctgccaggggccactgttaccctgatctgccctgggaaggaagcagcaggcaatgctaccattcactgggtgtactcaggctcacagagcagagaatggactaccacgggaaacacactggttctgagggccgtgcaggtcaatgacactgggcactatttgtgcttcctggatgatcatctggttgggactgtgcccttgctggtggatgttcccccagaggagcccaagctctcctgcttccggaagaacccccttgtaaatgccttttgtgagtggcatccaagcagcactccctctccaaccacgaaggctgtgatgtttgcaaagaaaatcaacaccaccaatgggaagagtgacttccaggtgccttgccagtattctcagcagctgaaaagcttctcctgcgaggtggagatcctggagggtgacaaagtgtaccacatagtgtcactgtgcgttgcaaacagtgtcggaagcaggtccagccacaatgtagtatttcagagtttaaaaatggtgcagccggatccacctgccaaccttgtggtatcagccatacctggaagcctcgttggctcaaagtcagttggcaagaccctgagtcctgggacccaagttactacttgttgcaattcgagcttcgataccgacctgtatggtcaaagaacgttcacggtgtggccgctccaggtggcccagcatcaatgtgtcatccatgatgccttgcgaggagtaaagcatgtggtgcaggtccgagggaaggaggagtttgacattggccagtggagcaaatggtccccggaggtcacaggcactccttggctagcagagcccaggaccactccggcagggatcccggggaaccccacacaggtctctgttgaagactatgacaaccacgaggatcagtacggaagttctacagaagcaacgagtgtcctcgccccagtgcaaggatcctcgcctatacccctgcccacattcctggtagctggaggaagcctggcgtttggattgcttctctgtgtcttcatcatcttgagactcaagaagaaatggaagtcacaggctgagaaggaaagcaagacgacttctcccccaccgtatcccttgggaccgctgaagccgaccttcctcctggttcctctcctcaccccatcagggtcccataacagctctgggactgacaacaccggaagccacagctgcctgggtgtcagggacccacagtgccctaatgacaacagcaacagagactacttattccccagataa

SEQ ID NO: 10 - protein, 364 aa, Rattus norvegicus
MLAVGCTLLVALLAAPAVALVLGSCRALEVANGTVTSLPGATVTLICPGKEAAGNATIHWVYSGSQSREWTTTGNTLVLRAVQVNDTGHYLCFLDDHLVGTVPLLVDVPPEEPKLSCFRKNPLVNAFCEWHPSSTPSPTTKAVMFAKKINTTNGKSDFQVPCQYSQQLKSFSCEVEILEGDKVYHIVSLCVANSVGSRSSHNVVFQSLKMVQPDPPANLVVSAIPGSLVGSKSVGKTLSPGTQVTTCCNSSFDTDLYGQRTFTVWPLQVAQHQCVIHDALRGVKHVVQVRGKEEFDIGQWSKWSPEVTGTPWLAEPRTTPAGIPGNPTQVSVEDYDNHEDQYGSSTEATSVLAPVQGSSPIPLP

SEQ ID NO: 11 - DNA, 1407 bp, Human
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatag

SEQ ID NO: 12 - protein, 365 aa, Human
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLP

(19) United States
(12) Patent Application Publication
(10) Pub. No.: US 2008/0145367 A1
(43) Pub. Date: Jun. 19, 2008

(54) Methods of Treating Osteoarthritis with Il-6 Antagonists

(75) Inventors: Susan Elizabeth Bove, Ann Arbor, MI (US); Kenneth S. Kilgore, King of Prussia, PA (US)

Correspondence Address: PFIZER INC., PATENT DEPARTMENT, MS8260-1611, GROTON, CT, 06340, US

(73) Assignee: Warner-Lambert Compay, LLC

(21) Appl. No.: 10/588,911

(22) PCT Filed: Jan. 31, 2005
(86) PCT No.: PCT/IB05/00240

§ 371 Date: Aug. 9, 2006

Related U.S. Application Data
(60) Provisional application No. 60/543,814, filed on Feb. 11, 2004.

(51) Int. Cl. A61K 39/395; A61P 19/02

(52) U.S. Cl. 424/141.1; 424/130.1

(57) ABSTRACT

The present invention provides for methods of treating osteoarthritis with IL-6 antagonists such as IL-6 antibodies.

### BACKGROUND OF THE INVENTION

Osteoarthritis is a disease that affects millions of people. Osteoarthritis patients suffer from symptoms such as joint pain and joint stiffness leading to joint deformities and diminishment or loss of joint function. Aspirin and conventional nonsteroidal anti-inflammatory drugs (NSAIDs) such as ibuprofen, diclofenac, and naproxen, are typical agents used to treat osteoarthritis sufferers. There is a need in the art for additional methods of treating osteoarthritis with therapeutic agents.

### SUMMARY OF THE INVENTION

In one aspect, the present invention relates to methods of treating osteoarthritis comprising: administering, to a subject suffering from a osteoarthritis, a pharmaceutical composition comprising a pharmaceutically acceptable carrier and a therapeutically effective amount of one or more agents selected from the group consisting of: an anti-IL-6 antibody and an anti-IL-6 receptor antibody. In certain embodiments the pharmaceutical composition is administered interarticularly or intravenously. In certain embodiments, the IL-6 receptor antibody and the IL-6 receptor antibody are monoclonal antibodies. In certain embodiments, the IL-6 receptor antibody is tocilizumab. In other embodiments, the IL-6 antibody is CNTO 328. In certain embodiments, the present invention relates to further administering one or more agents selected from the group consisting of: 6-(5-carboxy-5-methyl-hexyloxy)-2,2-dimethyl-hexanoic acid calcium salt, non-steroidal anti-inflammatory agents, piroxicam, diclofenac, naproxen, flurbiprofen, fenoprofen, ketoprofen, ibuprofen, mefenamic acid, indomethacin, sulindac, apazone, phenylbutazone, aspirin, celecoxib, parecoxib, valdecoxib, etoricoxib, corticosteroids, hyalgan, and synvisc. In certain embodiments, osteoarthritic pain may be treated with an anti-IL-6 antibody or an anti-IL-6 receptor antibody. In certain embodiments, the present invention relates to methods of treating osteoarthritis comprising: administering, to a subject suffering from a osteoarthritis, a pharmaceutical composition comprising a pharmaceutically acceptable carrier and a therapeutically effective amount of an anti-IL-6 antibody. In certain embodiments, the present invention relates to methods of treating osteoarthritis comprising: administering, to a subject suffering from a osteoarthritis, a pharmaceutical composition comprising a pharmaceutically acceptable carrier and a therapeutically effective amount of an anti-IL-6 receptor antibody.

In another aspect, the present invention relates to the use of one or more agents selected from the group consisting of: an anti-IL-6 antibody and an anti-IL-6 receptor antibody, in the manufacture of a medicament for the treatment of osteoarthritis in subjects.

### DEFINITIONS

In a clinical setting, a physician may assess whether a patient is suffering from osteoarthritis by standard clinical indices, including radiological methods (e.g., x-rays of affected joints), and determination of The Western Ontario and McMaster Universities Osteoarthritis Index (“WOMAC”) (see e.g., Creamer et al. (1999) J. Rheumatol. 26: 1785-1792).

The term “antibody” refers to a monomeric (e.g., single chain antibodies) or multimeric polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term “antibody” also includes antigen-binding polypeptides such as Fab, Fab′, F(ab′)₂, Fd, Fv, dAb, and complementarity determining region (CDR) fragments, single-chain antibodies (scFv), chimeric antibodies, and diabodies. The term antibody includes polyclonal antibodies and monoclonal antibodies unless otherwise indicated.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The amino terminus of each chain defines a variable region of about 100 to 110 or more amino acids primarily responsible for antigen recognition. The terms variable light chain (V_{L}) and variable heavy chain (V_{H}) refer to these light and heavy chains respectively.

As used herein, a Fd fragment means an antibody fragment that consists of the V_{H} and C_{H}1 domains; an Fv fragment consists of the V_{L} and V_{H} domains of a single arm of an antibody; and a dAb fragment (Ward et al., Nature 341:544-546 (1989)) consists of a V_{H} domain.

In some embodiments, the antibody is a single-chain antibody (scFv) in which a V_{L} and V_{H} domains are paired to form a monovalent molecule via a synthetic linker that enables them to be made as a single protein chain. (Bird et al., Science 242:423-426 (1988) and Huston et al., Proc. Natl. Acad. Sci. USA 85:5879-5883 (1988).) In some embodiments, the antibodies are diabodies, i.e., are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. (See e.g., Holliger P. et al., Proc. Natl. Acad. Sci. USA 90: 6444-6448 (1993), and Poljak R. J. et al., Structure 2:1121-1123 (1994)).

An “anti-IL-6” antibody is an antibody that specifically binds an IL-6 polypeptide. Examples of IL-6 polypeptides include, but are not limited to, a mouse IL-6 polypeptide (e.g., SEQ ID NO: 2), a rat IL-6 polypeptide (e.g., SEQ ID NO: 4), and a human IL-6 polypeptide (e.g., SEQ ID NO: 6). An example of an “anti-IL-6 antibody” is CNTO 328 (cCLB8), a human-mouse chimeric monoclonal antibody to IL-6 (see e.g., van Zaanen, et al. (1998) Br. J. Haematol. 102: 783-790).

An “anti-IL-6-receptor antibody” is an antibody that specifically binds the extracellular domain of an IL-6 receptor polypeptide. An example of an “anti-IL-6 receptor antibody” is MRA (tocilizumab). Examples of IL-6R extracellular domain polypeptides include, but are not limited to, a mouse IL-6R polypeptide (e.g., SEQ ID NO: 8), a rat IL-6R polypeptide (e.g., SEQ ID NO: 10), and a human IL-6R polypeptide (e.g., SEQ ID NO: 12).

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide antigen, refers to a binding reaction that is determinative of the presence of a specified protein. Typically, an antibody specifically binds an antigen when it has a K_{d} of at least about 1 μM or lower, more usually at least about 0.1 μM or lower, and preferably at least about 10 nM or lower for that antigen.

A variety of immunoassay formats (e.g., Western blots, ELISAs, etc.) may be used to select antibodies specifically immunoreactive with a particular protein. For example, solid-phase ELISA immunoassays are routinely used to select antibodies specifically immunoreactive with a protein (see, e.g., Harlow and Lane, Antibodies: A Laboratory Manual, New York: Cold Spring Harbor Press, (1990) for a description of immunoassay formats and conditions that can be used to determine specific immunoreactivity).

As used herein, the term “human antibody” means any antibody in which the variable and constant domain sequences are human sequences. The term encompasses antibodies with sequences derived from human genes, but which have been changed, e.g. to decrease possible immunogenicity, increase affinity, eliminate cysteines that might cause undesirable folding, etc. The term encompasses such antibodies produced recombinantly in non-human cells, which might impart glycosylation not typical of human cells. These antibodies may be prepared in a variety of ways, as described below.

The term “chimeric antibody” as used herein means an antibody that comprises regions from two or more different antibodies. In one embodiment, one or more of the CDRs are derived from a human anti-IL-6 antibody. In another embodiment, all of the CDRs are derived from a human anti-IL-6 antibody. In another embodiment, the CDRs from more than one human anti-IL-6 antibodies are combined in a chimeric antibody. For instance, a chimeric antibody may comprise a CDR1 from the light chain of a first human anti-IL-6 antibody, a CDR2 from the light chain of a second human anti-IL-6 antibody and a CDR3 from the light chain of a third human anti-IL-6 antibody, and the CDRs from the heavy chain may be derived from one or more other anti-IL-6 antibodies. Further, the framework regions may be derived from one of the anti-IL-6 antibodies from which one or more of the CDRs are taken or from one or more different human antibodies. For example, one or more CDRs from a non-human species (e.g., mouse or rat) antibody may be recombinantly inserted into a human antibody framework resulting in a “humanized” antibody.

### DETAILED DESCRIPTION

The present invention relates to methods of treating a subject suffering from osteoarthritis by administering a therapeutically effective amount of an anti-IL-6 antibody or an anti-IL-6 receptor antibody. Methods have been described for generating IL-6 antibodies (see e.g., Wendling et al. (1993) J. Rheumatol. 20: 259-262; U.S. Pat. No. 5,618,700), including humanized anti-human IL-6 antibodies (see e.g., U.S. Pat. Nos. 6,121,423 and 5,856,135), and IL-6R antibodies (see e.g., U.S. Pat. Nos. 5,795,965 and 5,817,790); MRA (tocilizumab; atilzumab; rhPM-1 (Drugs of the Future (2003) 28: 314-319) (Chugai Pharmaceutical Co., Ltd.) which was derived from the mouse anti-human IL-6R antibody PM1 (see e.g., Hirata et al. (1999) J. Immunol. 143: 2900-2906).

For preparation of IL-6 and IL-6R monoclonal or polyclonal antibodies, technique knowns in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). In addition, phage display technology can be used to identify single chain antibodies and heteromeric Fab fragments that specifically bind to selected antigens (see, e.g., McCafferty et al, Nature 348:552-554 (1990); Marks et al., Biotechnology 10:779-783 (1992)). Typically IL-6 and IL-6R polypeptides are employed to generate IL-6 and IL-6R antibodies, respectively. In the case of IL-6 polypeptides, they can be purified from native sources, cells that naturally secrete IL-6 polypeptides. Alternatively, synthetic peptides derived from IL-6 and IL-6R sequences disclosed herein and conjugated to a carrier protein can be used as an immunogen. In addition, recombinant IL-6 or IL-6R polypeptides can be employed to generate cognate antibodies. For example, recombinant mouse IL-6 (Catalog No. 406-ML-025), rat IL-6 (Catalog No. 506-RL-050) and human IL-6 (Catalog No. 206-IL-010) polypeptides as well as a recombinant soluble extracellular domain human IL-6R polypeptide (Catalog No. 227-SR-025) are commercially available from R&D Systems Inc., Minneapolis, Minn. In addition, nucleic acids encoding IL-6 (see e.g., Hirano et al. (1986) Nature 324: 73-76; Brakenhoff et al. (1987) J. Immunol. 139: 4116-4121; SEQ ID NOS: 1, 3, and 5) and IL-6R (see e.g., Yamasaki et al. (1988) Science 241: 825-828; SEQ ID NOS: 7, 9, and 11) can be made or isolated using routine techniques in the field of recombinant genetics and synthetic nucleic acid chemistry. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Current Protocols in Molecular Biology, Ausubel et al., eds., 1998.

Polyclonal antibodies typically can be generated by immunization of an animal with the antigen of choice. The immunization of the animals can be by any method known in the art. See, e.g., Harlow and Lane, Antibodies: A Laboratory Manual, New York: Cold Spring Harbor Press, 1990. Methods for immunizing non-human animals such as mice, rabbits, rats, sheep, goats, pigs, cattle and horses are well known in the art. See, e.g., Harlow and Lane, supra, and U.S. Pat. No. 5,994,619.

In certain embodiments, an IL-6 antigen is administered with an adjuvant to stimulate the immune response. Exemplary adjuvants include complete or incomplete Freund's adjuvant, RIBI (muramyl dipeptides) or ISCOM (immunostimulating complexes). Preferably, if a polypeptide is being administered, the immunization schedule will involve two or more administrations of the polypeptide, spread out over several weeks.

After immunization of an animal with an IL-6 or an IL-6R antigen, polyclonal antibodies and/or antibody-producing cells can be obtained from the animal. In some embodiments, anti-IL-6 or anti-IL-6R antibody-containing serum is obtained from the animal by bleeding or sacrificing the animal. The serum may be used as it is obtained from the animal, an immunoglobulin fraction may be obtained from the serum, or the anti-IL-6 or anti-IL-6R antibodies may be purified from the serum.

The animal's immune response to an immunogen preparation can be monitored by taking test bleeds and determining the titer of reactivity to the protein of choice. When appropriately high titers of antibody to the immunogen are obtained, blood can be collected from the animal and antisera are prepared. The level of IL-6 or IL-6R antibodies in serum can be assayed using an IL-6 or an IL-6R immunoassay. The polyclonal antibodies can be purified from the serum of an immunized animal using standard antibody and protein purification techniques.

Monoclonal antibodies can also be prepared against IL-6 and IL-6R. In certain embodiments, hybridoma techniques can be used to generate monoclonal antibodies. For example, antibody-producing immortalized cell lines can be prepared from cells isolated from the immunized animal. After immunization, the animal is sacrificed and lymph node and/or splenic B cells are immortalized. Methods of immortalizing cells include, but are not limited to, transfecting them with oncogenes, infecting them with an oncogenic virus, cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line).

Immortalized cells can be screened using IL-6 or IL-6R, or portions thereof, or a cell expressing IL-6 or IL-6R. In certain embodiments, the initial screening can be performed using an enzyme-linked immunoassay (ELISA) or a radioimmunoassay.

In some embodiments, human antibodies are produced by immunizing a non-human animal comprising in its genome some or all of human immuno-globulin heavy chain and light chain loci with an IL-6 or an IL-6R antigen. In certain embodiments, the non-human animal can be a XENOMOUSE™ animal (Abgenix Inc., Fremont, Calif.). Another non-human animal that may be used is a HuMAb-Mouse®, a transgenic mouse produced by Medarex (Medarex, Inc., Princeton, N.J.).

XENOMOUSE™ mice are engineered mouse strains that comprise large fragments of human immunoglobulin heavy chain and light chain loci and are deficient in mouse antibody production. See, e.g., Green et al., Nature Genetics 7:13-21 (1994) and U.S. Pat. Nos. 5,916,771, 5,939,598, 5,985,615, 5,998,209, 6,075,181, 6,091,001, 6,114,598, 6,130,364, 6,162,963 and 6,150,584. The splenic B cells from a XENOMOUSE™ can be fused to a non-secretory mouse myeloma (e.g, the myeloma cell line P3-X63-AG8-653) and monoclonal antibodies may be identified from the resulting pool of hybridomas. The IL-6 or IL-6R antibodies secreted by a hybridoma may be purified from a hybridoma culture and used in the methods of the present invention. The nucleic acids encoding the heavy and light chains of the IL-6 or IL-6R antibody may be isolated from a hybridoma and expressed in a host cell, e.g., NSO cells, CHO cells etc., to provide a source material from which purified IL-6 or IL-6 antibodies may be obtained.

In another embodiment, a transgenic animal is immunized with IL-6 or IL-6R, primary cells, e.g., spleen or peripheral blood cells, are isolated from an immunized transgenic animal and individual cells producing antibodies specific for the desired antigen are identified. Polyadenylated mRNA from each individual cell is isolated and reverse transcription polymerase chain reaction (RT-PCR) is performed using sense primers that anneal to variable region sequences, e.g., degenerate primers that recognize most or all of the FR1 regions of human heavy and light chain variable region genes and antisense primers that anneal to constant or joining region sequences. The cDNAs of the heavy and light chain variable regions are then cloned and expressed in any suitable host cell, e.g., a myeloma cell, as chimeric antibodies with respective immunoglobulin constant regions, such as the heavy chain and κ, or δ constant domains. See Babcook, J. S. et al., Proc. Natl. Acad. Sci. USA 93:7843-48, 1996, herein incorporated by reference. Anti IL-6 or IL-6R antibodies may then be identified and isolated as described herein.

In another aspect, the invention provides a method for making humanized anti-IL-6 or anti-IL-6R antibodies. In some embodiments, rats or mice are immunized with an IL-6 or an IL-6R antigen as described below under conditions that permit antibody production. Antibody-producing cells are isolated from the animals, fused with myelomas to produce hybridomas, and nucleic acids encoding the heavy and light chains of an anti-IL-6 or an anti-IL-6R antibody of interest are isolated. These nucleic acids are subsequently engineered using techniques known to those of skill in the art and as described further below to reduce the amount of non-human sequence, i.e., to humanize the antibody to reduce the immune response in humans

In another embodiment, phage display techniques can be used to provide libraries containing a repertoire of antibodies with varying affinities for IL-6 or IL-6R. By way of example, one method for preparing the library of antibodies for use in phage display techniques comprises the steps of immunizing a non-human animal comprising human immunoglobulin loci with an IL-6 or an IL-6R polypeptide to create an immune response, extracting antibody producing cells from the immunized animal; isolating RNA from the extracted cells, reverse transcribing the RNA to produce cDNA, amplifying the cDNA using a primer, and inserting the cDNA into a phage display vector such that antibodies are expressed on the phage. The resulting phage are tested for immunoreactivity to an IL-6 or IL-6R polypeptide. Recombinant anti-IL-6 or anti-IL-6R antibodies of the invention may be obtained in this way.

Techniques for the identification of high affinity human antibodies from such libraries are described for example in U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology 9:1370-1372 (1991); Hay et al., Hum. Antibod. Hybridomas 3:81-85 (1992); Huse et al., Science 246:1275-1281 (1989); McCafferty et al., Nature 348:552-554 (1990); Griffiths et al., EMBO J. 12:725-734 (1993); Hawkins et al., J. Mol. Biol. 226:889-896 (1992); Clackson et al., Nature 352:624-628 (1991); Gram et al., Proc. Natl. Acad. Sci. USA 89:3576-3580 (1992); Garrad et al., Bio/Technology 9:1373-1377 (1991); Hoogenboom et al., Nuc. Acid Res. 19:4133-4137 (1991); and Barbas et al., Proc. Natl. Acad. Sci. USA 88:7978-7982 (1991).

There are commercially available kits for generating phage display libraries (e.g., the Pharmacia Recombinant Phage Antibody System, catalog no. 27-9400-01; and the Stratagene SurfZAP™ phage display kit, catalog no. 240612) as well as commercially available systems for producing fully human phage expressed antibodies such as Cambridge Antibody Technology PLC (Cambridge, United Kingdom) and MorphoSys AG (e.g., HuCAL® GOLD technology, Martinsried, Germany).

Following screening and isolation of an anti-IL-6 or an anti-IL-6R antibody from a recombinant immunoglobulin display library, nucleic acids encoding the selected antibody can be recovered from the display package (e.g., from the phage genome) and subcloned into other expression vectors by standard recombinant DNA techniques. For example, the DNA encoding a phage expressed antibody can be cloned into a recombinant expression vector and introduced into a mammalian host cells or prokaryotic cells as appropriate for that antibody.

### Pharmaceutical Compositions

The invention also relates to pharmaceutical compositions comprising an anti-IL-6 or anti-IL-6R antibody for the treatment of subjects in need of treatment for osteoarthritis. Treatment may involve administration of one or more anti-IL-6 or anti-IL-6R monoclonal antibodies of the invention, alone or with a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, isotonic agents, for example, sugars, polyalcohols such as mannitol, sorbitol, or sodium chloride can be present in the composition. Additional examples of pharmaceutically acceptable substances are wetting agents or minor amounts of auxiliary substances such as wetting or emulsifying agents, preservatives or buffers, which enhance the shelf life or effectiveness of the antibody.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The particular form depends on the intended mode of administration and therapeutic application. Typical compositions are in the form of injectable or infusible solutions, such as compositions similar to those used for passive immunization of humans.

Therapeutic compositions typically are sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-IL-6 or anti-IL-6R antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation include vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the antibody composition may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

### Therapeutic Methods of Use

In another embodiment, the invention provides for methods for treating a subject suffering from osteoarthritis by administering a therapeutically effective amount of an anti-IL-6 or an anti-IL-6R antibody to a subject in need thereof. A “therapeutically effective amount” refers to an amount, at dosages and for periods of time necessary, sufficient to inhibit, halt, or allow an improvement in the disorder or condition being treated when administered alone or in conjunction with another pharmaceutical agent or treatment in a particular subject or subject population. The term “subject” refers to a member of the class Mammalia. Examples of mammals include, without limitation, humans, primates, chimpanzees, rodents, mice, rats, rabbits, horses, dogs, cats, sheep, and cows. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

It should be appreciated that the determination of proper dosage forms, dosage amounts, and routes of administration is within the level of ordinary skill in the pharmaceutical and medical arts. A therapeutically effective amount of the antibody may vary according to factors such as the disease state, age, sex, and weight of the individual, and the ability of the antibody to elicit a desired response in the individual. A therapeutically effective amount is also one in which any toxic or detrimental effects of an agent are outweighed by the therapeutically beneficial effects.

The antibody may be administered once or multiple times. For example, the antibody may be administered from three times daily to once every six months or longer. The administering may be on a schedule such as three times daily, twice daily, once daily, once every two days, once every three days, once weekly, once every two weeks, once every month, once every two months, once every three months and once every six months.

Co-administration of an antibody with an additional therapeutic agent (combination therapy) encompasses administering a pharmaceutical composition comprising the anti-IL-6 or anti-IL-6R antibody and the additional therapeutic agent and administering two or more separate pharmaceutical compositions, one comprising the anti-IL-6 or anti-IL-6R antibody and the other(s) comprising the additional therapeutic agent(s). Further, co-administration or combination therapy refers to antibody and additional therapeutic agents being administered at the same time as one another, as wells as instances in which an antibody and additional therapeutic agents are administered at different times. For instance, an antibody may be administered once every three days, while the additional therapeutic agent is administered once daily. Alternatively, an antibody may be administered prior to or subsequent to treatment of the disorder with the additional therapeutic agent. An antibody and one or more additional therapeutic agents (the combination therapy) may be administered once, twice or at least the period of time until the condition is treated, palliated or cured.

For example, anti-IL-6 and/or IL-6R antibodies may be co-administered with agents such as TNF-α antibodies such as REMICAD™, CDP-870 and HUMIR™, TNFα receptor immunoglobulin fusion molecules (such as ENBRE™), COX-2 inhibitors (such as celecoxib, rofecoxib, parecoxib, valdecoxib, and etoricoxib), metalloprotease-13 inhibitors (preferably MMP-13 selective inhibitors), non-steroidal anti-inflammatory agents (“NSAIDs”) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, 6-(5-carboxy-5-methyl-hexyloxy)-2,2-dimethyl-hexanoic acid, calcium salt (gemcabene calcium), α2δ ligands (such as NEUROTIN™ AND PREGABALIN™), and intraarticular therapies such as corticosteroids and hyaluronic acids such as hyalgan and synvisc.

The antibodies of the present invention can be administered by a variety of methods known in the art including, via an oral, mucosal, buccal, intranasal, inhalable, intravenous, subcutaneous, intramuscular, parenteral, or topical route. In certain embodiments, the mode of administration is parenteral (e.g., intravenous, subcutaneous, intraperitoneal, intramuscular). In certain embodiments, the antibody is administered by intravenous infusion or injection. In particular embodiment, the antibody is administered by intrarticular, intramuscular or subcutaneous injection. As will be appreciated by the skilled artisan, the route and/or mode of administration will vary depending upon the desired results.

Dosage regimens can be adjusted to provide the optimum desired response (e.g., a therapeutic response). For example, a single bolus can be administered, 2005/080429 PCT/IB2005/000240 several divided doses can be administered over time or the dose can be proportionally reduced or increased as indicated by the exigencies of the therapeutic situation. Parenteral compositions can be formulated in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier

An exemplary, non-limiting range for a therapeutically effective amount of an antibody of the invention from 1 to 40 mg/kg. In certain embodiments, the dose is 8-20 mg. In other embodiments, the dose is 10-12 mg. In certain embodiments, a dose range for intrarticular injection would be a 15-30 mg/dose. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### EXAMPLES 1-3

### Materials and Methods

Anti-IL-6 antibodies and anti-IL-6 receptor antibodies can be assayed for their ability to decrease quantitative or qualitative markers in in vivo models of osteoarthritis. For example, a monosodium iodoacetate-induced model of osteoarthritis (see e.g., Bove et al. (2003) Osteoarthritis and Cartilage 11: 821-830) can be carried out in rats to assess the effect of IL-6 antibodies in a weight bearing assay.

In Examples 1-3 on Day 0 rats are anesthetized with isofluorine, and the right, hind leg knee joint of a male Wistar rat is injected with 1.0 mg of mono-iodoacetate (“MIA”) in 50 μl phosphate buffered saline (PBS) through the infrapatellar ligament and the left, hind leg knee joint is injected with 50 μl of saline through the infrapatellar ligament. The injection of MIA into the joint results in the inhibition of glycolysis and eventual death of surrounding chondrocytes. On the day before antibody administration, Day 6 or Day 13 post-MIA injection, the hind-paw weight differential between the arthritic right hind joint and the saline injected left hind joint of male Wistar rats (150 g) is determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw.

The rats are then further administered via intra-articular injection or intraperitoneally, with 50 μl PBS containing 1, 3, 10, 20, or 30 μg of either a polyclonal goat anti-rat IL-6 antibody (R&D Systems Inc., Minneapolis, Minn.), or a polyclonal anti-rat IgG antibody (Product No. R 5005, Sigma, St. Louis, Mo.) on day 7 or day 14 post MIA-injection and the hind-paw weight differential is measured at 0-24 hours post antibody injection.

The percent inhibition of a change in hind paw joint function is calculated as the percent change in hind-paw weight distribution for treated animals versus control animals at the same time point (e.g., polyclonal anti-IL-6 antibody versus polyclonal anti-IgG antibody at 2 hours post injection). For example,

$$Percent inhibition of a change in hind paw weight distribution = \{ 1 - [ \frac{( Δ {W}_{G} )}{( Δ {W}_{C} )} ] \} \times 100$$

wherein:

ΔW_{C} is the hind-paw weight differential between the healthy left limb and the arthritic limb of the control animal administered the anti-rat IgG antibody alone, as measured at a particular time point (e.g., 1, 4, or 24 hours) post injection Day 7 or Day 14; and

ΔW_{G} is the hind-paw weight differential between the healthy left limb and the arthritic limb of the animal administered the anti-rat IL-6 antibody, as measured at the same time point used to determine ΔW_{C}.

### EXAMPLE 1

The MIA model was carried out as described above under Materials and Methods, as follows: rats were induced with MIA as described above, and administered 1, 3, 10, 20, or 30 μg of the polyclonal IL-6 antibody or the polyclonal IgG antibody in the right arthritic knee in a 50 μl volume of PBS and 501 μl volume of PBS in the left control knee on day 7 post-MIA injection. Six rats were injected at each dose. After one-hour post-antibody injection, the weight differential was measured. The percent inhibition of a change in hind paw weight distribution of the IL-6 antibody treated rats as compared to the polyclonal IgG antibody treated rats is reported in Table 1. The 20 and 30 microgram doses of IL-6 antibody significantly inhibited (p<0.05) the change in hind paw weight distribution versus polyclonal rat IgG. Data are presented as the mean percent inhibition ± standard error of the mean (SEM).

TABLE 1

| Dose | |
|---|---|
| (μg/knee) | % Inhibition |
| 1 | 28 ± 5 |
| 3 | 27 ± 12 |
| 10 | 18 ± 8 |
| 20 | 60 ± 7* |
| 30 | 63 ± 4* |

*p < 0.05 vs. polyclonal rat IgG (One-Factor ANCOVA followed by Hochberg's procedure)

### EXAMPLE 2

The MIA model was carried out as described above under Materials and Methods, as follows: rats were induced with MIA as described above, and administered 30 μg of the IL-6 antibody in the right arthritic knee in a 50 μl volume of PBS and 50 μl volume of PBS in the left control knee on day 14 post-MIA injection. Eight rats were injected at each dose. After one hour, 4 hours, and 24 hours post-antibody injection, the weight differential was measured and reported as the mean ± the standard error of the mean in Table 2. The 30 microgram dose of IL-6 antibody significantly decreased (p<0.05) the change in hind paw weight distribution at 1, 4, and 24 hours versus time zero (pre-antibody injection).

TABLE 2

| Time post- | |
|---|---|
| injection of | Weight differential |
| antibody | (grams) |
| (hours) | (Mean ΔW_{G} ± SEM) |
| 0 | 33 ± 3 |
| 1 | 17 ± 2* |
| 4 | 19 ± 2* |
| 24 | 17 ± 1* |

*p < 0.05 vs. time zero (paired t-test followed by Hochberg's procedure)

### EXAMPLE 3

The MIA model was carried out as described above under Materials and Methods, as follows: rats were induced with MIA as described above, and administered 30 μg of the IL-6 antibody via an intraperitoneal injection in a 50 μl volume of PBS on day 14 post-MIA injection. Eight rats were injected at each dose. The weight differential (ΔW_{G}) was measured and reported as the mean ΔW_{G}+± the standard error of the mean in Table 3 for the time points of just prior to antibody injection, at one hour, and at 4 hours post-antibody injection. The 30 microgram dose of IL-6 antibody did not significantly inhibit the change in hind paw weight distribution versus time zero (pre-antibody injection).

TABLE 3

| Time post- | |
|---|---|
| injection of | Weight differential |
| antibody | (grams) |
| (hours) | (Mean ΔW_{G} ± SEM) |
| 0 | 32 ± 2 |
| 1 | 28 ± 1 |
| 4 | 32 ± 2 |

It is understood that the examples and embodiments described herein are for illustrative purposes only and that various modifications or changes in light thereof will be suggested to persons skilled in the art and are to be included within the spirit and purview of this application and the scope of the appended claims. All publications, patents, and patent applications cited herein are hereby incorporated by reference in their entirety for all purposes.

What is claimed is:

1. A method of treating osteoarthritis comprising:
administering, to a subject suffering from a osteoarthritis, a pharmaceutical composition comprising a pharmaceutically acceptable carrier and a therapeutically effective amount of one or more agents selected from the group consisting of: an anti-IL-6 antibody and an anti-IL-6 receptor antibody.

2. The method of claim 1, wherein said pharmaceutical composition is administered interarticularly or intravenously.

3. The method of claim 1, wherein said agent is a monoclonal IL-6 receptor antibody.

4. The method of claim 3, wherein said IL-6 receptor antibody is an anti-human IL-6 receptor antibody.

5. The method of claim 3, wherein said IL-6 receptor antibody is tocilizumab.

6. The method of claim 1, wherein said agent is a monoclonal IL-6 antibody.

7. The method of claim 6, wherein said IL-6 antibody is an anti-human IL-6 antibody.

8. The method of claim 6, wherein said IL-6 antibody is CNTO 328.

9. The method of claim 6, wherein said pharmaceutical composition is administered interarticularly.

10. The method of claim 6, wherein said pharmaceutical composition is administered intravenously.

11. The method of claim 1, further comprising administering one or more agents selected from the group consisting of:
6-(5-carboxy-5-methyl-hexyloxy)-2,2-dimethyl-hexanoic acid calcium salt, non-steroidal anti-inflammatory agents, piroxicam, diclofenac, naproxen, flurbiprofen, fenoprofen, ketoprofen, ibuprofen, mefenamic acid, indomethacin, sulindac, apazone, phenylbutazone, aspirin, corticosteroids, hyalgan, and synvisc.

12. The method of claim 1, further comprising administering one or more agents selected from the group consisting of:
parecoxib, celecoxib, valdecoxib, and etoricoxib.

13. The use of one or more agents selected from the group consisting of: an anti-IL-6 antibody and an anti-IL-6 receptor antibody, in the manufacture of a medicament for the treatment of osteoarthritis in mammals.
